(19) United States
(12) Patent Application Publication
(10) Pub. No.: US 2024/0307578 A1
(43) Pub. Date: Sep. 19, 2024

(54) Aromatherapy Device

(71) Applicant: SHENZHEN LISEN INTELLIGENT CO., LTD., Shenzhen (CN)

(72) Inventor: Rufeng Yang, Shenzhen, Guangdong (CN)

(73) Assignee: SHENZHEN LISEN INTELLIGENT CO., LTD., Shenzhen (CN)

(21) Appl. No.: 18/234,886

(22) Filed: Aug. 17, 2023

(30) Foreign Application Priority Data
Mar. 17, 2023 (CN) 202320551774.8

(51) Int. Cl. A61L 9/12

(52) U.S. Cl. CPC A61L 9/12

(57) ABSTRACT

The present invention provides an aromatherapy device, the aromatherapy device is installed in an aromatherapy placement seat, the aromatherapy device comprises a supporting device, and the supporting device comprises an aromatherapy installation position, a shell and a movable limiting piece; the aromatherapy installation position is configured to install an aromatherapy volatile piece; the limiting piece has a first state and a second state, when the limiting piece is in the first state, the limiting piece protrudes from the shell to be abutted against the corresponding aromatherapy placement seat, to limit shaking of the aromatherapy device; and when the limiting piece is in the second state, the limiting piece is retracted to a position that does not protrude from the shell, such that an aromatherapy machine is detached from the aromatherapy placement seat.

### CROSS REFERENCE TO RELATED APPLICATIONS

The present application claims the benefit of Chinese Patent Application No. 202320551774.8 filed on Mar. 17, 2023, the contents of which are incorporated herein by reference in their entirety.

### TECHNICAL FIELD

The present invention relates to the technical field of aromatherapy, in particular to an aromatherapy device.

### BACKGROUND ART

With an improvement in life quality, in some places with strong smells, such as cars and other environments, aromatherapy devices are usually used to purify air and improve comfortable feelings of people in turn. The working principle of the aromatherapy device is as follows: essential oils and other fragrances are decomposed or volatilized, and gas produced during volatilization will be emitted to the environment, to adjust the smell of air.

The existing aromatherapy device is generally placed in an aromatherapy placement seat on an automobile, while in a process in which an automobile moves, the aromatherapy device often shakes relative to the aromatherapy placement seat, then the aromatherapy device is not placed stably in the aromatherapy placement seat, and noise will be produced when the aromatherapy device shakes and collides onto the aromatherapy placement seat, thereby influencing use experience of a user.

### SUMMARY OF THE INVENTION

The present invention provides an aromatherapy device, to solve the problems that the aromatherapy device is not stable and will produce noise when the aromatherapy device shakes in an aromatherapy placement seat.

To solve the above technical problem, the present invention provides an aromatherapy device. The aromatherapy device is installed in an aromatherapy placement seat, the aromatherapy device includes a supporting device, the supporting device includes an aromatherapy installation position, a shell and a movable limiting piece; and the aromatherapy installation position is configured to install an aromatherapy volatile piece; and the limiting piece has a first state and a second state, when the limiting piece is in the first state, the limiting piece protrudes from the shell to be abutted against the corresponding aromatherapy placement seat, to limit shaking of the aromatherapy device; and when the limiting piece is in the second state, the limiting piece is retracted to a position that does not protrude from the shell, such that an aromatherapy machine is detached from the aromatherapy placement seat.

According to an embodiment of this invention, the supporting device further comprises a rotating piece, a transmission piece and the limiting piece, the rotating piece and the transmission piece form a linkage structure, in the linkage structure, a rotary motion of the rotating piece can be converted into a linear motion of the transmission piece along an axial direction of the rotating piece; the transmission piece is connected with the limiting piece, and the linear motion of the transmission piece can be converted into the linear motion of the limiting piece along a radial direction of the rotating piece, such that the limiting piece can protrude along the radial direction of the rotating piece, to abut against the aromatherapy placement seat and limit the aromatherapy device.

According to an embodiment of this invention, the shell is provided with a guiding channel, the guiding channel extends along the radial direction of the rotating piece, the limiting piece moves along the guiding channel, and when the transmission piece moves along the axial direction of the rotating piece, the guiding channel limits the limiting piece, and guides the limiting piece to move along the guiding channel.

According to an embodiment of this invention, a side wall of the shell is formed with a cut-through first opening, a bottom end of the first opening extends to a bottom wall of the shell, and the first opening and the bottom wall of the shell form the guiding channel.

According to an embodiment of this invention, a first guiding structure is arranged on the bottom wall of the shell, a second guiding structure is arranged on a surface, facing towards the bottom wall of the shell, of the limiting piece, and the second guiding structure is configured to be in sliding connection with the first guiding structure.

According to an embodiment of this invention, the rotating piece is provided with a rotating part and a threaded rod, the rotating part is provided with the aromatherapy installation position, one end of the threaded rod is arranged on the rotating part, the other end of the threaded rod is arranged in the shell and installed on the bottom wall of the shell, the rotating part can rotate to drive the threaded rod to rotate relative to the shell; the transmission piece is sleeved on the threaded rod, and threads on an inner wall of the transmission piece are engaged with treads on an outer wall of the threaded rod.

According to an embodiment of this invention, the supporting device further comprises connecting rods, and two ends of each connecting rod are respectively in rotary connection with the transmission piece and the limiting piece.

According to an embodiment of this invention, the connecting rod and the limiting piece form a transmission component, multiple transmission components are available, multiple transmission components are uniformly distributed around periphery of the transmission piece, and the connecting rods of each transmission component are in rotary connection with the transmission piece; two connecting rods are available in the transmission component, which are respectively a first connecting rod and a second connecting rod, the first connecting rod and the second connecting rod are arranged at intervals along an axis of the rotating piece, the first connecting rod, the second connecting rod, a side wall of the transmission piece and the limiting piece form a parallelogram.

According to an embodiment of this invention, the limiting piece is provided with a first cavity, a second opening is formed on a side, facing towards the threaded rod, of the cavity, the connecting rod is in rotary connection with an inner wall of the cavity through the second opening, a third opening is formed on a side wall, facing towards the rotating part, of the limiting piece, the third opening extends to the second opening and is communicated with the second opening, the connecting rod can be inserted into the third opening in a process of being close to the rotating part, and the connecting rod can withdraw from the third opening in a process of being far away from the rotating part.

According to an embodiment of this invention, the aromatherapy device further comprises an aromatherapy component, the aromatherapy component comprises an aromatherapy volatile piece, and the aromatherapy component is arranged on the aromatherapy installation position of the supporting device.

The present invention provides an aromatherapy device, and the aromatherapy device is installed in an aromatherapy placement seat, the aromatherapy device includes a supporting device, the supporting device includes an aromatherapy placement seat, a shell and a movable limiting piece; and the aromatherapy installation position is configured to install an aromatherapy volatile piece; and the limiting piece has a first state and a second state, when the limiting piece is in the first state, the limiting piece protrudes from the shell to be abutted against the corresponding aromatherapy placement seat, to limit shaking of the aromatherapy device; and when the limiting piece is in the second state, the limiting piece is retracted to a position that does not protrude from the shell, such that an aromatherapy machine is detached from the aromatherapy placement seat. When an aromatherapy device is placed in an automobile, the aromatherapy device can move to the limiting piece through the limiting piece in the supporting device until the limiting piece is abutted against the aromatherapy placement seat, such that the aromatherapy device will not shake in the aromatherapy placement seat and the noise caused by shaking of the aromatherapy device will not be produced. Moreover, when the aromatherapy device needs to supplement or replace the internal aromatherapy volatile piece, the aromatherapy device can move to the limiting piece through the limiting piece in the supporting device until the limiting piece is separated from the aromatherapy placement seat, so as to take out the aromatherapy device from the aromatherapy placement seat.

### BRIEF DESCRIPTION OF THE DRAWINGS

FIG. 1 is a structural schematic diagram of an aromatherapy device in a first state provided in an embodiment of the present invention;

FIG. 2 is a structural schematic diagram of an aromatherapy device in a second state provided in an embodiment of the present invention;

FIG. 3 is a schematic diagram of an exploded structure of FIG. 2;

FIG. 4 is a structural schematic diagram of a rotating piece, a transmission piece and a limiting piece in a first state provided in an embodiment of the present invention;

FIG. 5 is a structural schematic diagram of a rotating piece, a transmission piece and a limiting piece in a second state provided in an embodiment of the present invention;

FIG. 6 is a structural schematic diagram of a shell provided in an embodiment of the present invention;

FIG. 7 is a structural schematic diagram of a limiting piece provided in an embodiment of the present invention.

### DETAILED DESCRIPTION OF THE EMBODIMENTS

The present invention is described in further details below through specific embodiments in combination with accompanying drawings. Wherein similar elements in different implementations employ associated similar element numbers. In the following implementations, many details are described to enable the present invention to be better understood. However, those skilled in the art can easily recognize that some features may be omitted or replaced by other elements, materials or methods in different cases. In some cases, some related operations of the present invention are not shown or described in the specification, to avoid a core part of the present invention being overwhelmed by excessive description, it is not necessary for those skilled in the art to describe these related operations in details, and those skilled in the art will fully understand related operations from the description in the specification and general knowledge of the art.

In addition, the characteristics, operations or features described in the specification may be combined in any suitable form to form various implementations. Operational steps involved in various embodiments may also be sequentially exchanged or adjusted in a manner apparent to those skilled in the art. Therefore, the specification and drawings are merely intended to clearly describe a certain embodiment and are not intended to be a necessary composition and/or sequence.

The serial numbers assigned herein for the parts themselves, such as “first”, “second”, etc. are merely used to distinguish the described objects without any sequential or technical meanings. The terms “connection” and “linkage” mentioned in the present invention include direct and indirect connection (linkage) unless otherwise specified.

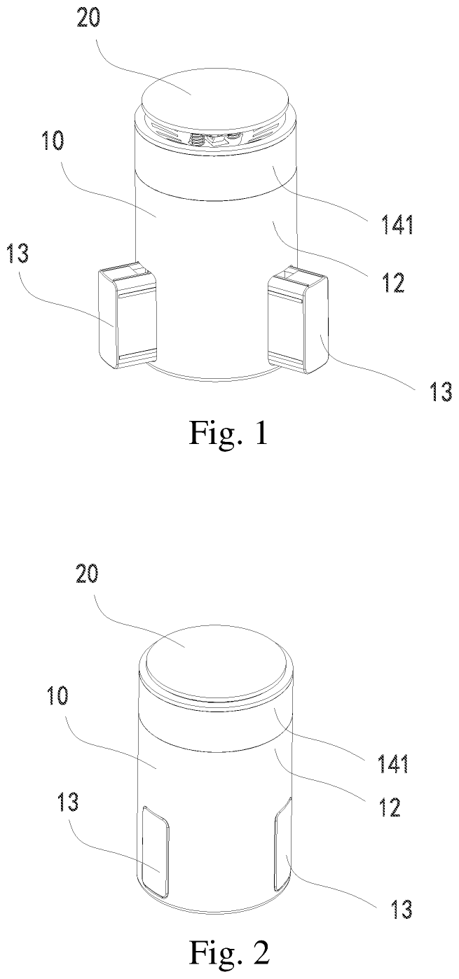

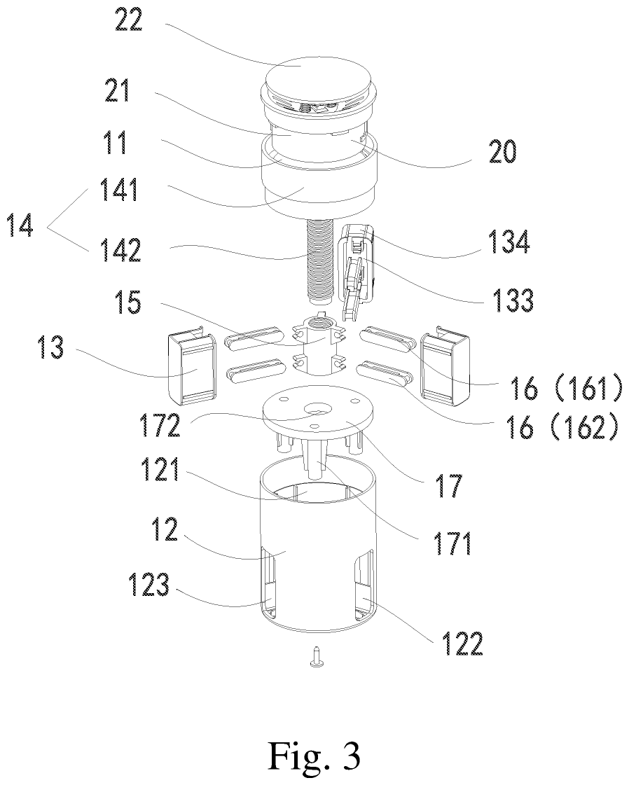

Please refer to FIG. 1 to FIG. 3, FIG. 1 is a structural schematic diagram of an aromatherapy device in a first state provided in an embodiment of the present invention, FIG. 2 is a structural schematic diagram of an aromatherapy device in a second state provided in an embodiment of the present invention, and FIG. 3 is a schematic diagram of an exploded structure of FIG. 2. The present invention provides an aromatherapy device, the aromatherapy device is installed in an aromatherapy placement seat (not shown in the figures), specifically, in one embodiment, the aromatherapy device is installed in an aromatherapy placement seat on an automobile. Or, in other embodiments, the aromatherapy device can also be installed in an aromatherapy placement seat of a moving object, for example, the aromatherapy device can also be installed in an aromatherapy placement seat on an elevator.

In one embodiment, the aromatherapy device includes a supporting device 10, and the supporting device 10 includes an aromatherapy installation position 11, a shell 12 and a movable limiting piece 13. Wherein the aromatherapy installation position 11 is configured to install an aromatherapy volatile piece (not shown in the figures), and the aromatherapy volatile piece can be for example, an aromatherapy liquid, an aromatherapy paste, an aromatherapy tablet, an aromatherapy block, or the like. The movable limiting piece 13 may be driven to move by an operator using an external force or may be driven to move by a driving mechanism (e.g., a motor).

In one embodiment, the limiting piece 13 has a first state and a second state, when the limiting piece 13 is in the first state, as shown in FIG. 1, the limiting piece 13 protrudes from the shell 12 to be abutted against the corresponding aromatherapy placement seat, to limit shaking of the aromatherapy device; for example, the limiting piece 13 can face towards the surface of the aromatherapy placement seat and can be abutted against an inner wall of the aromatherapy placement seat.

When the limiting piece 13 is in the second state, the limiting piece 13 is retracted to a position that does not protrude from the shell 12, such that an aromatherapy machine is detached from the aromatherapy placement seat. Specifically, an accommodating cavity 121 can be arranged inside the shell 12, a side wall of the shell 12 is provided with a cut-through first opening 122, and the limiting piece 13 can go into and out of the shell 12 through the first opening 122. The limiting piece 13 being retracted to a position that does not protrude from the shell 12 can mean that the limiting piece 13 is retracted to the inside of the accommodating cavity 121; or part of the limiting piece 13 is retracted to the inside of the accommodating cavity 121, while the other part can be flush with the first opening 122.

When the aromatherapy device of the present invention is placed in an automobile and other moving scenarios, since the aromatherapy device is installed in an aromatherapy placement seat on an automobile, the aromatherapy device can move to the limiting piece 13 through the limiting piece 13 in the supporting device 10 until the limiting piece 13 is abutted against the aromatherapy placement seat, such that the aromatherapy device will not shake in the aromatherapy placement seat, and the problem of noise caused when the aromatherapy device shakes will not occur. Moreover, when the aromatherapy device needs to supplement or replace internal aromatherapy volatile piece, the aromatherapy device can move to the limiting piece 13 through the limiting piece 13 in the supporting device 10 until the limiting piece 13 is separated from the aromatherapy placement seat, such that the aromatherapy device can be taken out from the aromatherapy placement seat.

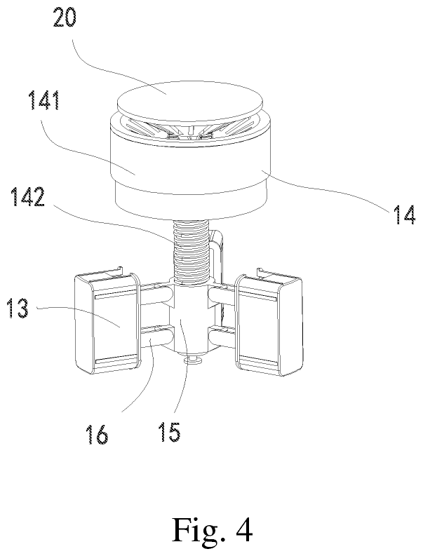

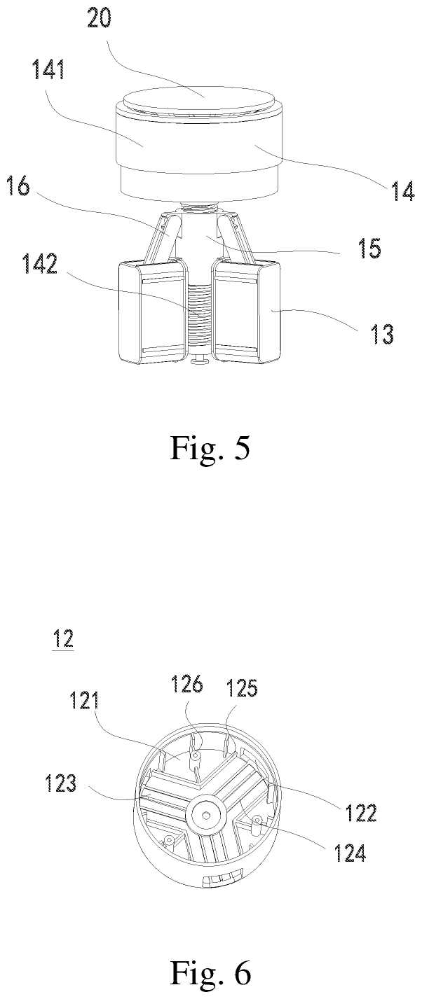

Please refer to FIG. 3, FIG. 4 and FIG. 5, FIG. 4 is a structural schematic diagram of a rotating piece 14, a transmission piece 15 and a limiting piece 13 in a first state provided in an embodiment of the present invention, and FIG. 5 is a structural schematic diagram of a rotating piece 14, a transmission piece 15 and a limiting piece 13 in a second state provided in an embodiment of the present invention. In one embodiment, the supporting device 10 further includes a rotating piece 14, a transmission piece 15 and a limiting piece 13. Transmission among the rotating piece 14, the transmission piece 15 and the limiting piece 13 can enable the limiting piece 13 to move, such that the limiting piece 13 is switched between the first state and the second state.

Specifically, the rotating piece 14 and the transmission piece 15 form a linkage structure. In the linkage structure, the rotary motion of the rotating piece 14 can be converted into a linear motion of the transmission piece 15 along an axial direction of the rotating piece 14, and an operator can manually rotate the rotating piece 14 to drive the rotating piece 14 to rotate along an axis of the rotating piece 14, such that the transmission piece 15 moves linearly along an axial direction of the rotating piece 14.

In one embodiment, please refer to FIG. 3 to FIG. 5, the linkage structure of the rotating piece 14 and the transmission piece 15 can be a screw nut transmission structure. Specifically, in one embodiment, the rotating piece 14 is provided with a rotating part 141 and a threaded rod 142. One side of the rotating part 141 is provided with an aromatherapy installation position 11, and the other side of the rotating part 141 is fixedly connected with the threaded rod 142. One end of the threaded rod 142 is arranged on the rotating part 141, the other end of the threaded rod 142 is arranged in the shell 12 and is installed on a bottom wall of the shell 12, and the rotating part 141 rotates to drive the threaded rod 142 to rotate relative to the shell 12. The transmission piece 15 is sleeved on the threaded rod 142, an inner wall of the transmission piece 15 is provided with threads, and threads on an inner wall of the transmission piece 15 are engaged with threads on an outer wall of the threaded rod 142. When the rotating part 141 rotates along a first direction, the first direction can be for example a clockwise direction or an anticlockwise direction, the transmission piece 15 can move linearly towards a direction deviating from the rotating part 141, when the rotating part 141 rotates along a direction opposite to the first direction, the transmission piece 15 can move linearly towards a direction close to the rotating part 141; that is, through alternately rotating the rotating part 141 in the first direction and the second direction, the transmission piece 15 can move linearly in a reciprocating manner on the threaded rod 142.

In one embodiment, the transmission piece 15 is connected with the limiting piece 13, wherein the transmission piece 15 can be directly or indirectly connected with the limiting piece 13. For example, in embodiments of FIG. 3 to FIG. 5, the supporting device 10 further includes a connecting rod 16, the connecting rod 16 indirectly connects the transmission piece 15 with the limiting piece 13, and two ends of the connecting rod 16 are respectively in rotary connection with the transmission piece 15 and the limiting piece 13.

In one embodiment, linear motion of the transmission piece 15 along an axial direction of the rotating piece 14 can be converted into linear motion of the limiting piece 13 along a radial direction of the rotating piece 14, such that the limiting piece 13 can protrude along a radial direction of the rotating piece 14, and a limiting piece 13 is abutted against the aromatherapy placement seat, to limit the aromatherapy device (as shown in FIG. 4); of course, linear motion of the transmission piece 15 can also be retracting of the limiting piece 13 along a radial direction of the rotating piece 14, to separate the limiting piece 13 from the aromatherapy placement seat (as shown in FIG. 5). Specifically, in embodiments of FIG. 3 to FIG. 5, reciprocating linear motion of the transmission piece 15 along an axial direction of the screw can be converted into reciprocating linear motion of the limiting piece 13 along a radial direction of the screw. Therefore, the supporting device 10 can enable the limiting piece 13 to perform reciprocating linear motion along a radial direction through rotating the rotating piece 14, such that the limiting piece 13 is abutted against or separated from the aromatherapy placement seat. Particularly, in embodiments of FIG. 3 to FIG. 5, the transmission piece 15 utilizes a pure mechanical structure to enable the limiting piece 13 to go into and out of the shell 12, the structure is simple and convenient for operation, and the cost is saved.

On one embodiment, please refer to FIG. 3 and FIG. 6. FIG. 6 is a structural schematic diagram of a shell 12 provided in an embodiment of the present invention. The shell 12 is provided with a guiding channel 123, and the guiding channel 123 extends along a radial direction of the rotating piece 14. The limiting piece 13 moves along the guiding channel 123, when the transmission piece 15 moves along an axial direction of the rotating piece 14, the guiding channel 123 limits the limiting piece 13, to guide the limiting piece 13 to move along the guiding channel 123. Through setting a guiding channel 123, when the transmission piece 15 moves, the transmission piece 15 is limited to drive the limiting piece 13 to move in an axial direction of the rotating piece 14, and the limiting piece 13 can also be guided to move along the guiding channel 123, such that the limiting piece 13 can move along a radial direction of the rotating piece 14.

In embodiments of FIG. 3 to FIG. 6, a side wall of the shell 12 is formed with a cut-through first opening 122, a bottom end of the first opening 122 extends to a bottom wall of the shell 12, and the first opening 122 and the bottom wall of the shell 12 form a guiding channel 123, to limit and guide the limiting piece 13. In other embodiments, the first opening 122 can also be arranged in a middle section of the side wall of the shell 12, and as a guiding channel 123, the first opening 122 limits and guides the limiting piece 13. Or, the limiting piece 13 also has two opposite surfaces along an axial direction of the rotating piece 14, an accommodating cavity 121 of the shell 12 is provided with a first limiting part and a second limiting part which extend along a radial direction of the rotating piece 14, a space between the first limiting part and the second limiting part is a guiding channel 123, the first limiting part and the second limiting part are respectively abutted against two surfaces of the limiting piece 13, to limit and guide the limiting piece 13. The setting of the guiding channel 123 is not limited to the cases mentioned above, and the guiding channel 123 can also be of other structures conceived by those skilled in the art.

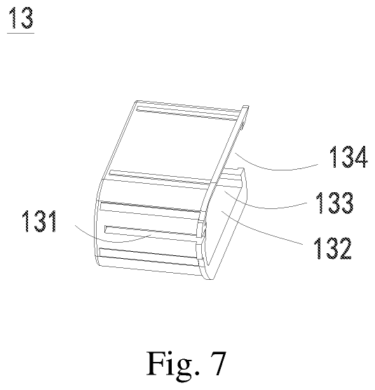

Further, in one embodiment, a guiding structure can also be arranged in the guiding channel 123, to guide a motion path of the limiting piece 13 along a radial direction of the rotating piece 14. As shown in FIG. 6 and FIG. 7, FIG. 7 is a structural schematic diagram of a limiting piece 13 provided in an embodiment of the present invention. A first guiding structure 124 is arranged on a bottom wall of the shell 12, a second guiding structure 131 is arranged on a surface, facing towards a bottom wall of the shell 12, of the limiting piece 13, and the second guiding structure 131 is configured to be in sliding connection with the first guiding structure 124. Wherein as shown in FIG. 6 and FIG. 7, one of the first guiding structure 124 and the second guiding structure 131 is a raised fillet, and the other is a guiding slot matched with the fillet, and the guiding slot is in sliding connection with the fillet. In addition, a guiding part 125 extending along a radial direction can also be arranged on a bottom wall of the shell 12, and the guiding part 125 is arranged at two sides of the side wall of the limiting piece 13, to guide movement of the limiting piece 13 in a radial direction at two sides of the side wall of the limiting piece 13.

In one embodiment, the connecting rod 16 and the limiting piece 13 form a transmission component. Multiple transmission components may be available, and the multiple transmission components are uniformly distributed around periphery of the transmission piece 15. For example, in embodiments of FIG. 3 to FIG. 6, three transmission components are available, and the three transmission components are uniformly distributed around periphery of the transmission piece 15. The connecting rod 16 of each transmission component is in rotating connection with the transmission piece 15. Moreover, the number of guiding channels 123 can be the same as the number of the transmission components, and the limiting pieces 13 of the transmission component are respectively arranged in corresponding guiding channels 123. For example, in embodiments of FIG. 3 to FIG. 6, three guiding channels 123 are available, that is, the number of the first opening 122 is three, and the number of the first guiding structure 124 is three. Through setting multiple transmission components, in the aromatherapy device, multiple limiting pieces 13 go into and out of the shell 12, and multiple limiting pieces 13 can be abutted against the corresponding aromatherapy placement seats, such that when the aromatherapy device is placed in the aromatherapy placement seat, the limiting piece 13 can be abutted against the aromatherapy placement seat more stably and reliably.

In one embodiment, as shown in FIG. 3 to FIG. 5, the number of connecting rods 16 in each transmission component is two, which are respectively a first connecting rod 161 and a second connecting rod 162, the first connecting rod 161 and the second connecting rod 162 are arranged at intervals along an axis of the rotating piece 14, two sides of the first connecting rod 161 and the second connecting rod 162 are all in rotary connection with the transmission piece 15 and the limiting piece 13, the first connecting rod 161, the second connecting rod 162, a side wall of the transmission piece 15 and the limiting piece 13 form a parallelogram. Through setting a parallelogram, transmission between the limiting piece 13 and the transmission piece 15 can be more stable.

In one embodiment, as shown in FIG. 4, FIG. 5 and FIG. 7, the limiting piece 13 is provided with a first cavity 132, a second opening 133 is formed on a side, facing towards the threaded rod 142, of the cavity 132, the connecting rod 16 is in rotary connection with an inner wall of the cavity 132 through the second opening 133, a third opening 134 is formed on a side wall, facing towards the rotating part 141, of the limiting piece 13, the third opening 134 extends to the second opening 133 and is communicated with the second opening 133, the connecting rod 16 can be inserted into the third opening 134 in a process of being close to the rotating part 141, and the connecting rod 16 can withdraw from the third opening 134 in a process of being far away from the rotating part 141. Therefore, as shown in FIG. 5, when the transmission piece 15 moves to be close to the rotating part 141, that is, when the limiting piece 13 is retracted into the shell 12, the connecting rod 16 can be at least partially accommodated in the cavity 132 of the limiting piece 13, to save space, such that the whole supporting device 10 can be small-sized, to satisfy the requirement of miniaturization of the aromatherapy device.

In one embodiment, as shown in FIG. 3, the supporting device 10 may also include a fixed piece 17, the fixed piece 17 covers on the top of the shell 12, and the fixed piece 17 can be matched with the second positioning column 126 in the accommodating cavity 121 through a first positioning column 171, to fix the fixed piece 17 on the shell 12. A bottom surface of the rotating part 141 of the rotating piece 14 is arranged on a side, deviating from the accommodating cavity 121, of the fixed piece 17, a screw of the rotating piece 14 penetrates through an open pore 172 on the fixed piece 17 and is arranged in the accommodating cavity 121, and the rotating piece 14 can rotate relative to the fixed piece 17 and the shell 12. Through detachable setting of the fixed piece 17 and the shell 12, assembly of the supporting component is facilitated.

In one embodiment, as shown in FIG. 1 to FIG. 5, the aromatherapy device further includes an aromatherapy component 20, the aromatherapy component 20 includes an aromatherapy volatile piece, and the aromatherapy component 20 is arranged on an aromatherapy installation position 11 of the supporting device 10. Further, the aromatherapy component 20 can further include an aromatherapy pot 21 configured to accommodate the aromatherapy volatile piece, and the aromatherapy pot 21 can also be provided with a cover body 22 for shielding the aromatherapy pot 21, and the cover body 22 can be opened and closed in a rotatable manner, or the cover body 22 can be opened and closed in a pressing manner, such that the cover body 22 can be opened when the aromatherapy device needs to be used, and the cover body 22 can be closed when the aromatherapy device does not need to be used.

The above description of the present invention with specific examples is merely intended to help understand the present invention, rather than limiting the present invention. For those skilled in the art to which the present invention belongs, a number of simple deductions, deformations or replacements can also be made according to ideas of the present invention.

What is claimed is:

1. An aromatherapy device, wherein the aromatherapy device is installed in an aromatherapy placement seat and comprises:
a supporting device, wherein the supporting device comprises an aromatherapy installation position, a shell and a movable limiting piece; and the aromatherapy installation position is configured to install an aromatherapy volatile piece; and
the limiting piece has a first state and a second state, when the limiting piece is in the first state, the limiting piece protrudes from the shell to be abutted against the corresponding aromatherapy placement seat, to limit shaking of the aromatherapy device; and when the limiting piece is in the second state, the limiting piece is retracted to a position that does not protrude from the shell, such that an aromatherapy machine is detached from the aromatherapy placement seat.

2. The aromatherapy device of claim 1, wherein the supporting device further comprises a rotating piece, a transmission piece and the limiting piece, the rotating piece and the transmission piece form a linkage structure, in the linkage structure, a rotary motion of the rotating piece can be converted into a linear motion of the transmission piece along an axial direction of the rotating piece; the transmission piece is connected with the limiting piece, and the linear motion of the transmission piece can be converted into the linear motion of the limiting piece along a radial direction of the rotating piece, such that the limiting piece can protrude along the radial direction of the rotating piece, to abut against the aromatherapy placement seat and limit the aromatherapy device.

3. The aromatherapy device of claim 2, wherein the shell is provided with a guiding channel, the guiding channel extends along the radial direction of the rotating piece, the limiting piece moves along the guiding channel, and when the transmission piece moves along the axial direction of the rotating piece, the guiding channel limits the limiting piece, and guides the limiting piece to move along the guiding channel.

4. The aromatherapy device of claim 3, wherein a side wall of the shell is formed with a cut-through first opening, a bottom end of the first opening extends to a bottom wall of the shell, and the first opening and the bottom wall of the shell form the guiding channel.

5. The aromatherapy device of claim 4, wherein a first guiding structure is arranged on the bottom wall of the shell, a second guiding structure is arranged on a surface, facing towards the bottom wall of the shell, of the limiting piece, and the second guiding structure is configured to be in sliding connection with the first guiding structure.

6. The aromatherapy device of claim 2, wherein
the rotating piece is provided with a rotating part and a threaded rod, the rotating part is provided with the aromatherapy installation position, one end of the threaded rod is arranged on the rotating part, the other end of the threaded rod is arranged in the shell and installed on the bottom wall of the shell, the rotating part can rotate to drive the threaded rod to rotate relative to the shell; the transmission piece is sleeved on the threaded rod, and threads on an inner wall of the transmission piece are engaged with treads on an outer wall of the threaded rod.

7. The aromatherapy device of claim 6, wherein the supporting device further comprises connecting rods, and two ends of each connecting rod are respectively in rotary connection with the transmission piece and the limiting piece.

8. The aromatherapy device of claim 7, wherein the connecting rod and the limiting piece form a transmission component, multiple transmission components are available, multiple transmission components are uniformly distributed around periphery of the transmission piece, and the connecting rods of each transmission component are in rotary connection with the transmission piece; two connecting rods are available in the transmission component, which are respectively a first connecting rod and a second connecting rod, the first connecting rod and the second connecting rod are arranged at intervals along an axis of the rotating piece, the first connecting rod, the second connecting rod, a side wall of the transmission piece and the limiting piece form a parallelogram.

9. The aromatherapy device of claim 7, wherein the limiting piece is provided with a first cavity, a second opening is formed on a side, facing towards the threaded rod, of the cavity, the connecting rod is in rotary connection with an inner wall of the cavity through the second opening, a third opening is formed on a side wall, facing towards the rotating part, of the limiting piece, the third opening extends to the second opening and is communicated with the second opening, the connecting rod can be inserted into the third opening in a process of being close to the rotating part, and the connecting rod can withdraw from the third opening in a process of being far away from the rotating part.

10. The aromatherapy device of claim 1, wherein the aromatherapy device further comprises an aromatherapy component, the aromatherapy component comprises an aromatherapy volatile piece, and the aromatherapy component is arranged on the aromatherapy installation position of the supporting device.
